(19) United States
(12) Patent Application Publication
(10) Pub. No.: US 2009/0274781 A1
(43) Pub. Date: Nov. 5, 2009

(54) ANTI-OBESITY AGENT

(75) Inventors: Takahiko Fujikawa, Tsu-Shi (JP); Sansei Nishibe, Sapporo-Shi (JP); Shigeharu Yamaguchi, Nagoya-Shi (JP); Koji Oba, Nagoya-Shi (JP)

Correspondence Address: BURR & BROWN, PO BOX 7068, SYRACUSE, NY, 13261-7068, US

(73) Assignee: ASGEN Pharmaceutical Co., Ltd., Nagoya-Shi (JP)

(21) Appl. No.: 12/261,277

(22) Filed: Oct. 30, 2008

(30) Foreign Application Priority Data
May 2, 2008 (JP) 2008-120128

(51) Int. Cl. A61K 36/752

(52) U.S. Cl. 424/736

(57) ABSTRACT

The present invention provides an anti-obesity agent having an anti-obesity effect obtained by combining a few extracts, which is attained by an anti-obesity agent containing Forsythia leaf extract, Citrus extract, and at least one extract selected from the group consisting of Licorice extract and Gardenia fruit extract, and it is preferable that the anti-obesity agent contains both Licorice extract and Gardenia fruit extract. The anti-obesity agent exhibits an ideal anti-obesity effect for increasing brown adipocytes and muscle weight to increase a basal metabolic rate and to decrease white adipocytes.

### BACKGROUND OF THE INVENTION

1) Field of the Invention

The present invention relates to an anti-obesity agent.

2) Description of the Related Art

Recently, it is said that diseases (for example, cardiac disease and diabetes mellitus and so on) caused by obesity are associated with many deaths. These diseases, which are often linked to lifestyle, are referred to as lifestyle-related diseases, and many lifestyle-related diseases have been known. Lifestyle-related disease is defined as a disease group in which lifestyles such as eating habits, fitness habits, rest, smoking, and drinking are involved in the symptoms and development thereof. There are known type 2 diabetes mellitus, obesity, hyperlipemia (excluding familial hyperlipemia), circulatory organ disease (excluding congenital circulatory organ disease) and hypertension and soon. Alternatively, attention has also been directed to metabolic syndrome in which a plurality of dangerous factors (abnormal glucose tolerance, hypertension, obesity, abnormalities in lipid metabolism and so on) overlap in the individual to develop deadly cardiovascular disease.

It is said that the number affected by patients of lifestyle-related diseases such as hypertension and hyperlipemia in conjunction with Westernization of lifestyles, accounts for 10 million to 20 million people including diabetics of 7 million or more in Japan. Thus, obesity is a serious social problem, and fights against obesity are not only a Japanese problem but also a global one.

Kampo medicines are multi-component-based medicines that have been widely used from a long time ago in Japan. A number of Kampo medicines have an effect of enhancing the natural healing energy of a living body which is difficult to confirm in Western medicines. However, the use of Kampo medicines is often based on experience, and the effect mechanism thereof may not be sufficiently clarified. One of the Kampo medicines having an anti-obesity effect is Bofutsushosan. Since the Bofutsushosan, which has a function of reducing body heat to dissipate the cause of disease, has an effect of improving fluid circulation in the body, Bofutsushosan is used for obesity, constipation, decrease in urinary volume, dropsy, dizziness and shoulder stiffness and so on (Patent Documents 1, 2).

Patent Document 1: Japanese Published Unexamined Patent Application No. 2007-277128

Patent Document 2: Japanese Published Unexamined Patent Application No. 2005-179316

### SUMMARY OF THE INVENTION

The present invention has been made in view of the above situation. It is an object of the present invention to provide an anti-obesity agent exhibiting an anti-obesity effect obtained by combining Forsythia leaf extract as a new component with a few components.

Through diligent study and research, the present inventor has found that an anti-obesity effect is exhibited by combining Forsythia leaf extract as a new component with Citrus extract, Licorice extract and Gardenia fruit extract and so on in order to confirm the effect of the Forsythia leaf extract. The present invention was fundamentally accomplished based on this finding.

Thus, an anti-obesity agent according to the present invention for attaining the above object includes Forsythia leaf extract, Citrus extract, and at least one extract selected from the group consisting of Licorice extract and Gardenia fruit extract.

It is preferable that the anti-obesity agent of the above invention includes both the Licorice extract and the Gardenia fruit extract.

Alternatively, it is preferable that the dosage form of the anti-obesity agent is any one of a tablet, a granule, a powdered medicine and a liquid medicine.

The “Forsythia leaf extract” means an extract from a Forsythia leaf. A method for extracting the Forsythia leaf extract is not necessarily limited. For example, a method for obtaining a typical crude drug extract can be used. The form of the Forsythia leaf extract is not limited. Examples of the form include powder, granule, grain and fluid etc. The Forsythia is a Chinese native oleaceous deciduous shrub (height: about 2 meters or so). The Forsythia has been cultivated in admiration for many, many years. The Forsythia, which has branches that extend, droops down the tips of the branches slightly. The fruits thereof are used as Forsythia fruit of crude drug, and have effects such as antiphlogistine, diuresis, drainage and detoxication. The Forsythia leaf means the leaf of the Forsythia. In the present invention, not the Forsythia fruits used for Kampo medicine but the Forsythia leaf is used.

The “Citrus extract” means an extract from Citrus. The method for extracting the Citrus extract is not necessarily limited. For example, a method of obtaining a typical crude drug extract can be used. The form of the Citrus extract is not limited. Examples of the form include powder, granule, grain and fluid etc. The Citrus means oranges, and means the general term for evergreens of mandarin oranges, particularly fruit trees and fruits. Taxonomically, the Citrus is divided into Citrus, Fortunella and Poncirus.

The “Licorice extract” means an extract from Licorice. A method for extracting the Licorice extract is not necessarily limited. For example, a method for obtaining a typical crude drug extract can be used. The form of the Licorice extract is not limited. Examples of the form include powder, granule, grain and fluid etc. The Licorice is a Leguminosae herbaceous perennial naturally growing in northeast to northwest China. The Licorice has a height of about a meter and has pinnate compound leaves. The root thereof, which is reddish-brown, is referred to as sweet root/licorice, and has a special sweet taste. It is used for analgesic and cough medicines as a crude drug.

The “Gardenia fruit extract” means an extract from gardenia. A method for extracting the Gardenia fruit extract is not necessarily limited. For example, a method for obtaining a typical crude drug extract can be used. The form of the Gardenia fruit extract is not limited. Examples of the form include powder, granule, grain and fluid etc. The Gardenia fruit is a rubiaceous evergreen shrub (height: one to three meters) described as gardenia and so on. The leaf thereof is subopposite, has a leather quality and is glossy, has a white six-petaled flower in summer, and has a strong aroma. When the fruits have ripened, they have a red and yellow color, and the yellow pigment taken therefrom has been used as a dye for many years. The dried fruits, which are used as Gardenia fruit of crude drug, and have effects such as sedation, homeostasis, anti-inflammation, diuresis and antipyresis.

The anti-obesity agent may also be obtained by respectively obtaining and then mixing the extracts, or the extract may also be obtained after mixing the respective components. Alternatively, each of the extracts may be formed into the shape of a tablet and so on, and an individually formed extract may be capable of being taken all together.

### BRIEF DESCRIPTION OF THE DRAWINGS

FIG. 1 is a graph obtained by investigating the influence on a food intake caused by MHE (four extracts containing Forsythia leaf extract, Citrus extract, Licorice extract, and Gardenia fruit extract) administration for three months. FIG. 1 shows data of a C1 group (negative control: ND H2O), A1 group (ND MHE 0.2%), A2 group (ND MHE 1%), A3 group (ND MHE 5%), C2 group (positive control: HFD H2O), B1 group (HFD MHE 0.2%), B2 group (HFD MHE 1%), and B3 group (HFD MHE 5%) from the left of the graph (the same even in FIGS. 2 to 16);

FIG. 2 is a graph obtained by investigating the influence on body weight caused by MHE administration for three months;

FIG. 3 is a graph obtained by investigating the influence on the weight of white adipocytes (WATr) surrounding the reins caused by MHE administration for three months;

FIG. 4 is a graph obtained by investigating the influence on the weight of white adipocytes (WATt) surrounding the testis caused by MHE administration for three months;

FIG. 5 is a graph obtained by investigating the influence on the weight of brown adipocytes (BAT) caused by MHE intake for three months;

FIG. 6 is a graph obtained by investigating the influence on the weight of soleus muscle (Sol.M.) caused by MHE administration for three months;

FIG. 7 is a graph obtained by investigating the influence on serum GOT levels caused by MHE administration for three months;

FIG. 8 is a graph obtained by investigating the influence on serum GPT levels caused by MHE administration for three months;

FIG. 9 is a graph obtained by investigating the influence on serum total cholesterol (T-CHO) levels caused by MHE administration for three months;

FIG. 10 is a graph obtained by investigating the influence on serum TG (triglyceride) levels caused by MHE administration for three months;

FIG. 11 is a graph obtained by investigating the influence on serum FFA (free fatty acid) levels caused by MHE administration for three months;

FIG. 12 is a graph obtained by investigating the influence on serum LDL-C (low-density cholesterol) levels caused by MHE administration for three months;

FIG. 13 is a graph obtained by investigating the influence on serum HDL-C (high-density cholesterol) levels caused by MHE administration for three months;

FIG. 14 is a graph obtained by investigating the influence on serum glucose levels caused by MHE administration for three months;

FIG. 15 is a graph obtained by investigating the influence on serum insulin levels caused by MHE administration for three months;

FIG. 16 is a graph obtained by investigating the influence on serum adiponectin levels caused by MHE administration for three months;

FIG. 17 is a graph obtained by investigating the influence on a food intake caused by the administration of an anti-obesity agent for four weeks. The left side and right side of the graph show data of the C group (positive control: HFD H2O) and D3 group (HFD MHE 5%) respectively;

FIG. 18 is a graph obtained by investigating the influence on a water intake mean caused by the administration of the anti-obesity agent for four weeks. FIG. 18 shows data of the C group (positive control: HFD H2O), D1 group (HFD FCGr 5%), D2 group (HFD FCGf 5%) and D3 group (HFD MHE 5%) from the left of the graph (the same even in FIGS. 19 to 22);

FIG. 19 is a graph obtained by investigating the influence on body weight caused by the administration of an anti-obesity agent for four weeks;

FIG. 20 is a graph obtained by investigating the influence on the weight of white adipocytes (WATr) surrounding the reins caused by the administration of the anti-obesity agent for four weeks;

FIG. 21 is a graph obtained by investigating the influence on the weight of white adipocytes (WATt) surrounding the testis caused by the administration of the anti-obesity agent for four weeks; and

FIG. 22 is a graph obtained by investigating the influence on the weight of brown adipocytes (BAT) caused by the administration of the anti-obesity agent for four weeks.

### DETAILED DESCRIPTION

An embodiment of the present invention will be described with reference to the drawings. However, the technical scope of the present invention is not limited by the embodiment. The present invention may be carried out in various forms without departing from the scope of the present invention.

### Example 1

### Preparation of Anti-Obesity Agent

(A) Forsythia leaf extract (Forsythia leaf extract produced by Tama Biochemical Co., Ltd.), (B) Citrus extract (Citrus Aurantium extract produced by ALPS Pharmaceutical Ind. Co., Ltd.), (C) Licorice extract (extract made from Licorice water produced by MIKUNI & CO., Ltd.), and (D) Gardenia fruit extract (dried Gardenia fruit extract produced by ALPS Pharmaceutical Ind. Co., Ltd.) were used for preparing the anti-obesity agent. All the above extracts (A) to (D) were powdered medicines. The following three kinds of anti-obesity agents were prepared.

An anti-obesity agent 1 having a mass ratio of (A):(B):(C)=3:3:5 was prepared.

An anti-obesity agent 2 having a mass ratio of (A):(B):(D)=1:1:1 was prepared.

An anti-obesity agent 3 having a mass ratio of (A):(B):(C):(D)=3:3:5:3 was prepared.

In the present specification, the anti-obesity agent 1, the anti-obesity agent 2 and the anti-obesity agent 3 are described as “FCGr,” “FCGf” and “MHE,” respectively in the specification.

### Example 2

### Effect Confirming Test of Anti-Obesity Agent 3 (MHE)

Four-week old male SD rats were divided into the following eight groups, and an effect confirming test of the MHE was carried out.

C1 group (negative control: ND H2O): The rats were made to freely take a normal diet (ND) containing 5% lard after preliminarily breeding the rats for one week. The rats were made to freely take water (H2O) as drinking water.

C2 group (positive control: HFD H2O): The rats were made to freely take a high fat diet (HFD) containing 35% lard after preliminarily breeding the rats for one week. The rats were made to freely take water (H2O) as drinking water.

A1 group (ND MHE 0.2%): The rats were made to freely take the ND containing the MHE of 0.2% after preliminarily breeding the rats for one week. The rats were made to freely take water as drinking water.

A2 group (ND MHE 1%): The rats were made to freely take the ND containing the MHE of 1% after preliminarily breeding the rats for one week. The rats were made to freely take water as drinking water.

A3 group (ND MHE 5%): The rats were made to freely take the ND containing the MHE of 5% after preliminarily breeding the rats for one week. The rats were made to freely take water as drinking water.

B1 group (HFD MHE 0.2%): The rats were made to freely take the HFD containing MHE of 0.2% after preliminarily breeding the rats for one week. The rats were made to freely take water as drinking water.

B2 group (HFD MHE 1%): The rats were made to freely take the HFD containing the MHE of 1% after preliminarily breeding the rats for one week. The rats were made to freely take water as drinking water.

B3 group (HFD MHE 5%): The rats were made to freely take the HFD containing MHE of 5% after preliminarily breeding the rats for one week. The rats were made to freely take water as drinking water.

Four rats were used per one group of the above C1 group and A1 to A3 groups, and six rats were used per one group of the above C2 group and B1 to B3 groups.

After breeding any of the above eight groups for three months, food intake mean (g/BW/day) per body weight per one day, body weight (g), a rate (WATr/BW (%)) of white adipocytes surrounding the reins per body weight, a rate (WATt/BW (%)) of white adipocytes surrounding the testis per body weight, a rate (BAT/BW (%)) of brown adipocytes per body weight, and a rate (Sol.M./BW (%)) of the soleus muscle per weight were measured.

Alternatively, as serum markers, serum GOT levels (IU/L), serum GPT levels (IU/L), serumtotalcholesterol (T-CHO) levels (mg/dL), serum TG (triglyceride) levels (mg/dL), serum FFA (free fatty acid) levels (uEq/L), serum LDL-C (low-density cholesterol) levels (mg/dL), serum HDL-C (high-density cholesterol) levels (mg/dL), serum glucose levels (mg/dL), serum insulin levels (gm/mL), and serum adiponectin levels (ng/mL) were measured.

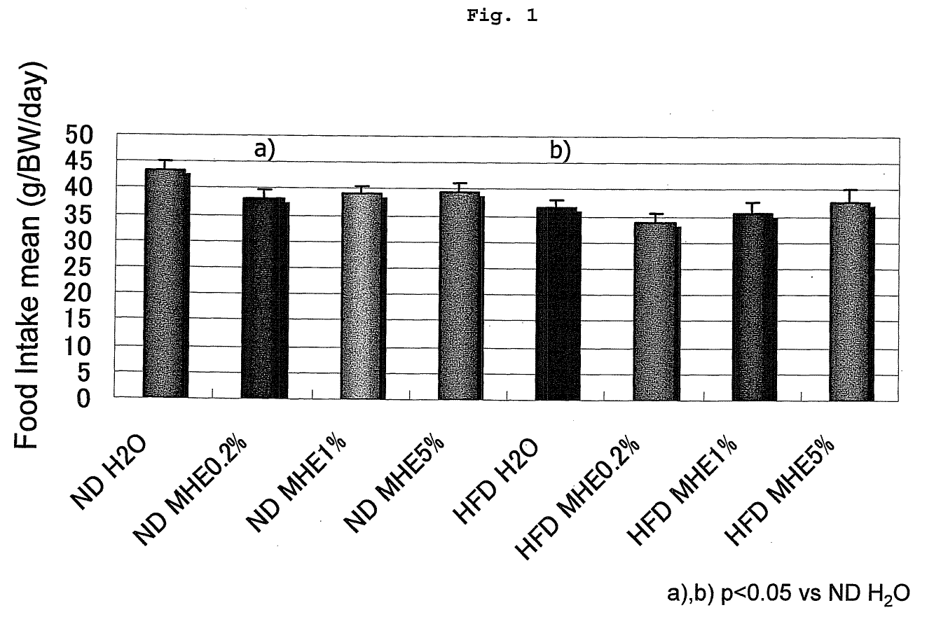

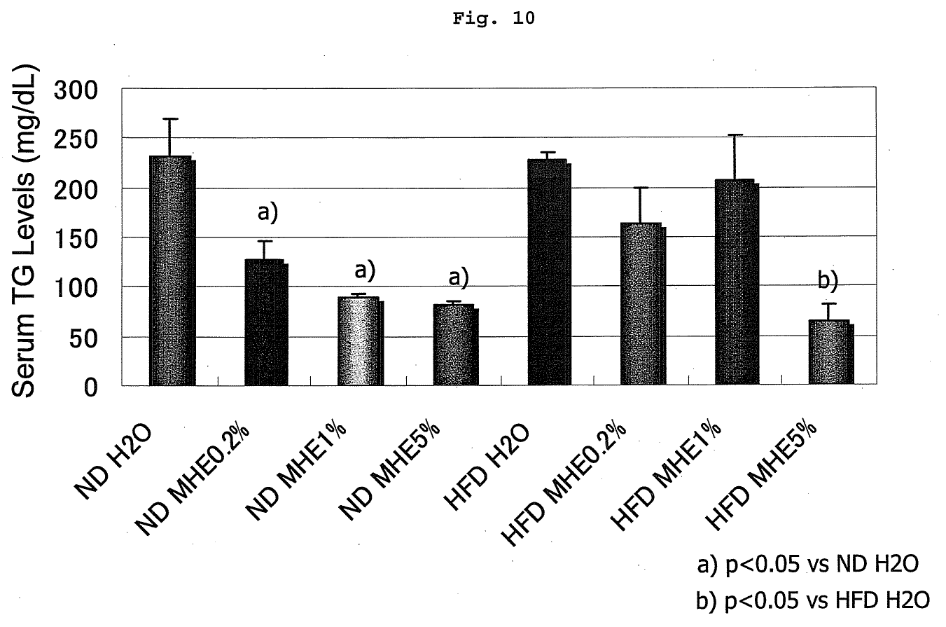

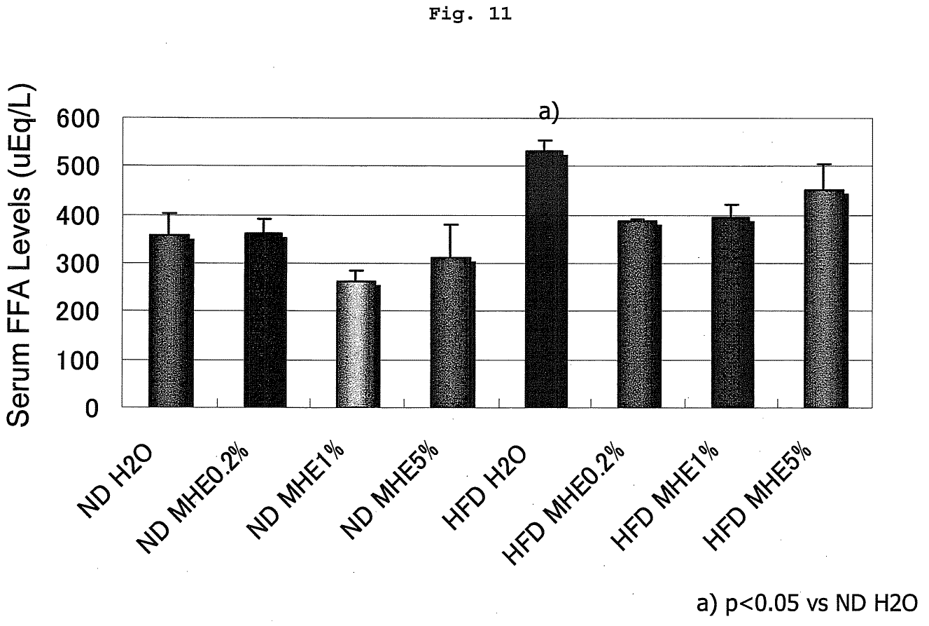

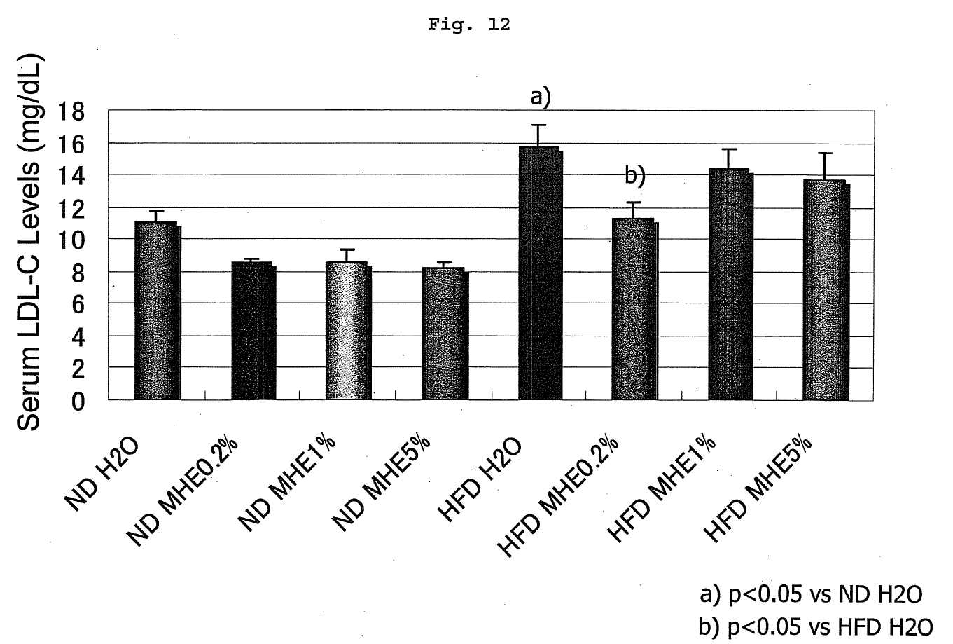

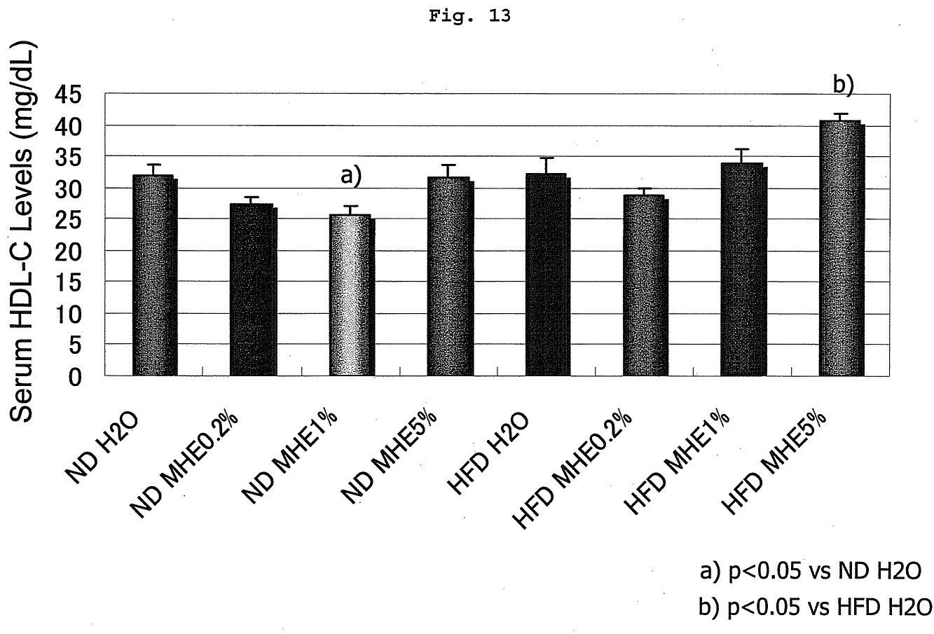

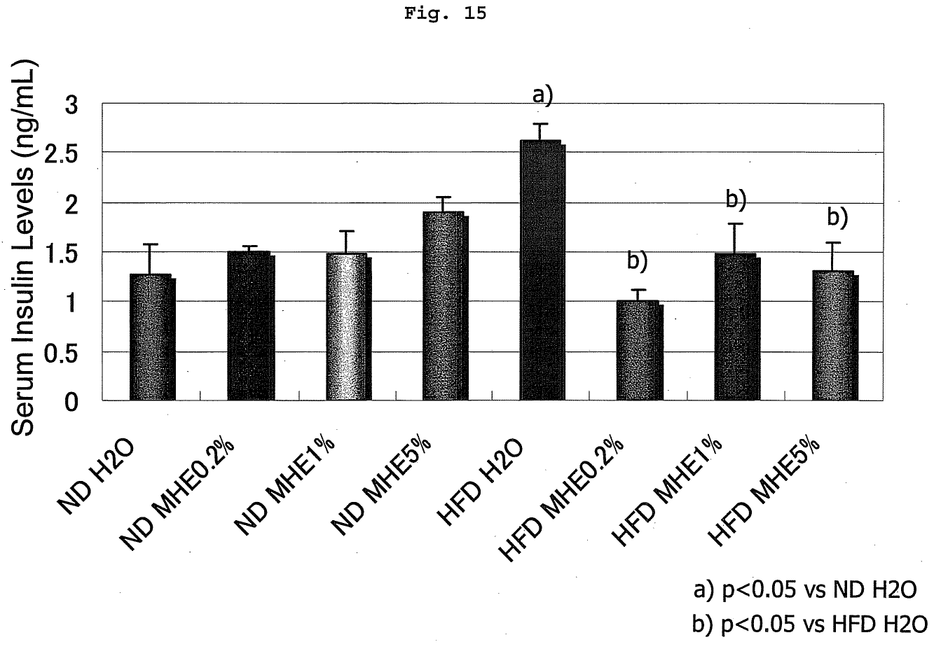

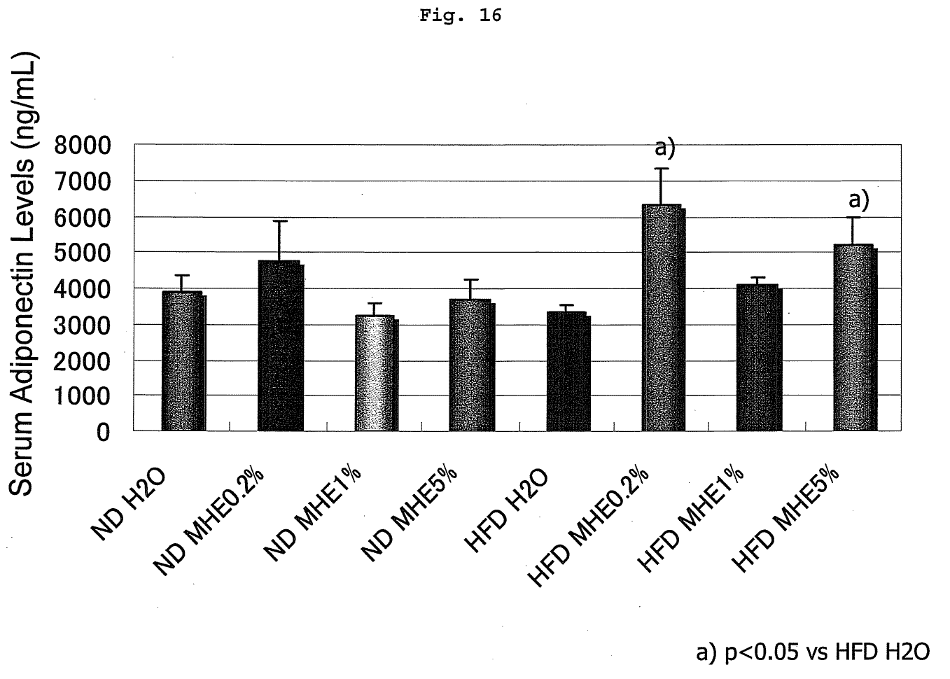

FIGS. 1 to 16 show the results of the example 2. Data in the figures are shown by an average value±standard error. Symbols (a) to (c) in FIGS. 1 to 16 mean that a significant difference (p<0.05) was observed between the C1 group (ND, H2O) or the C2 group (HFD, H2O).

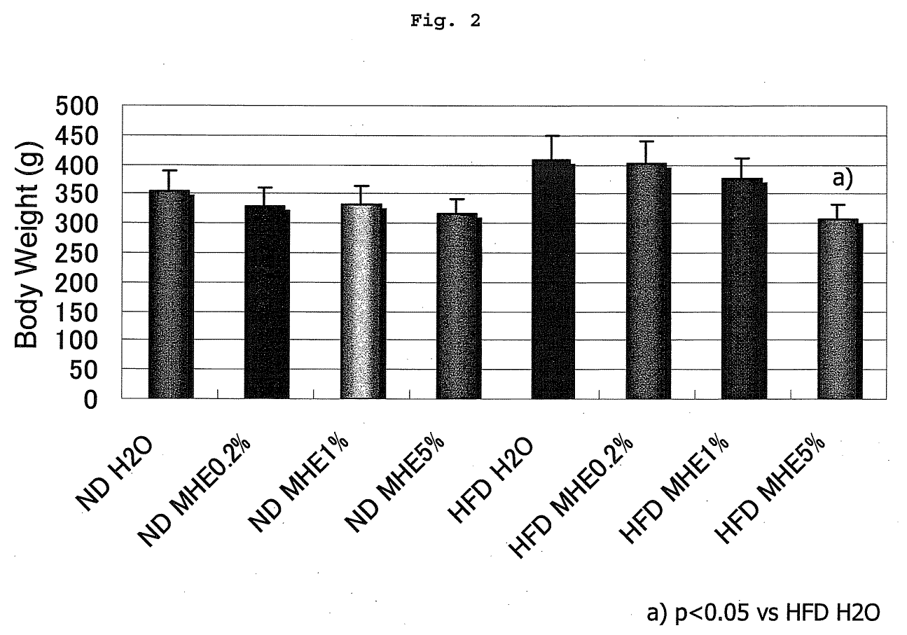

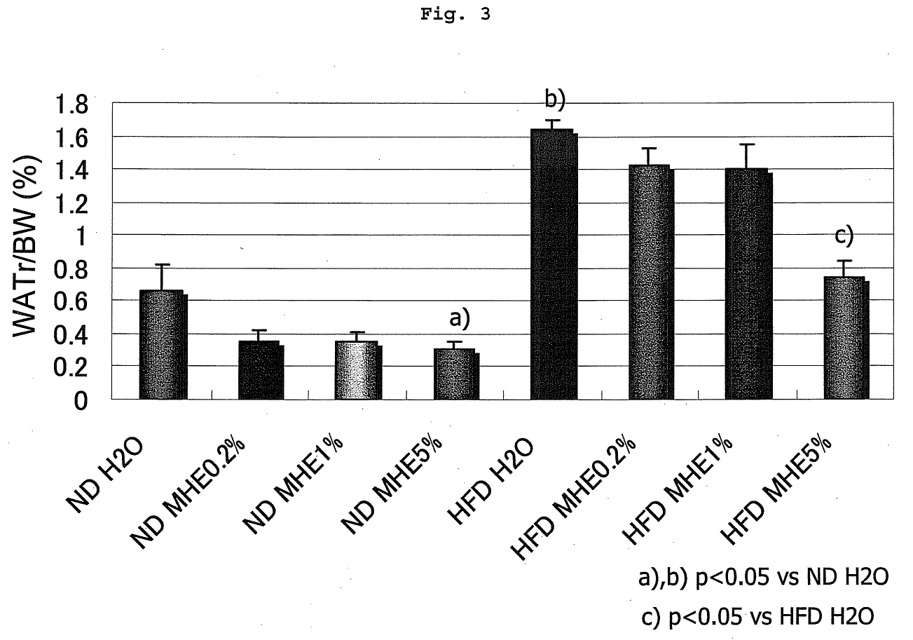

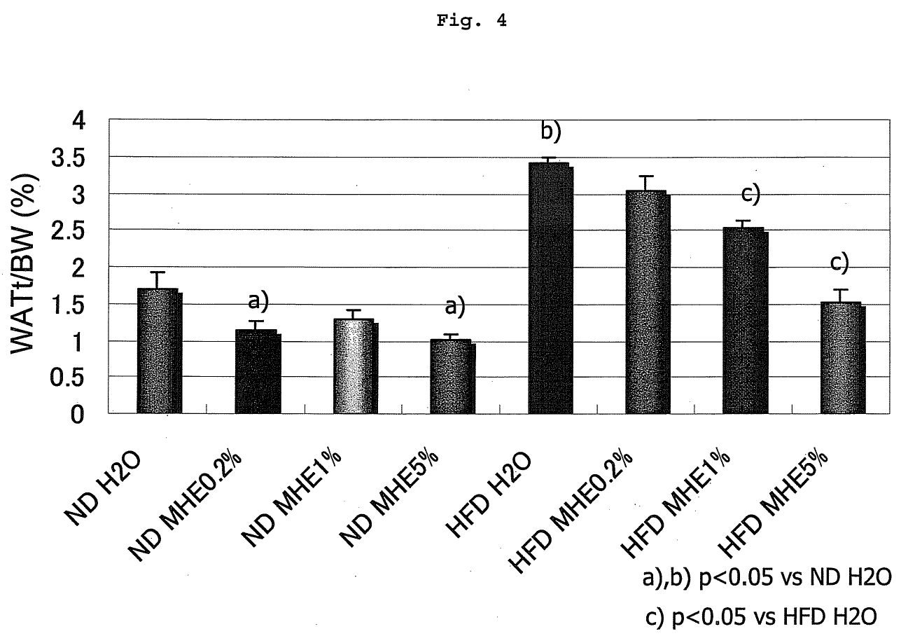

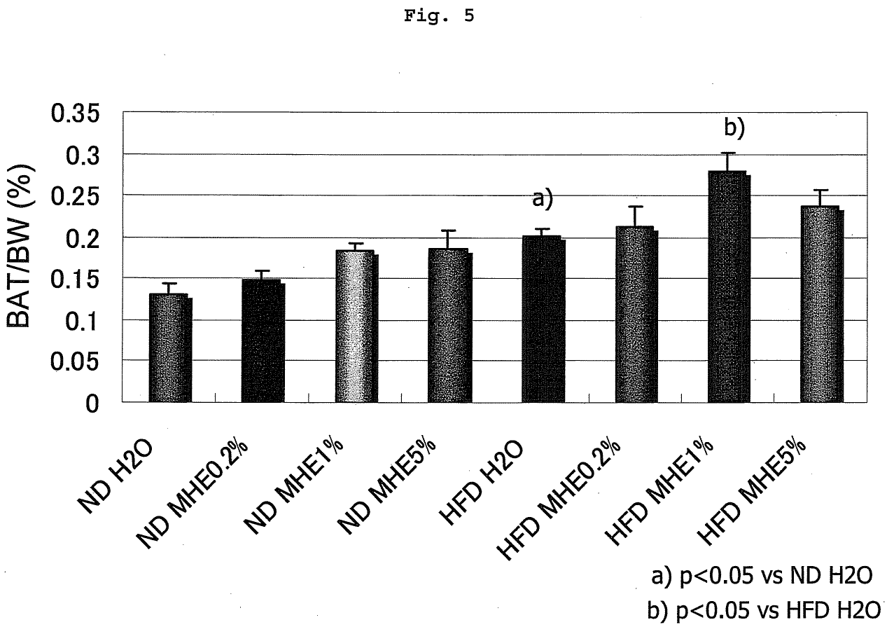

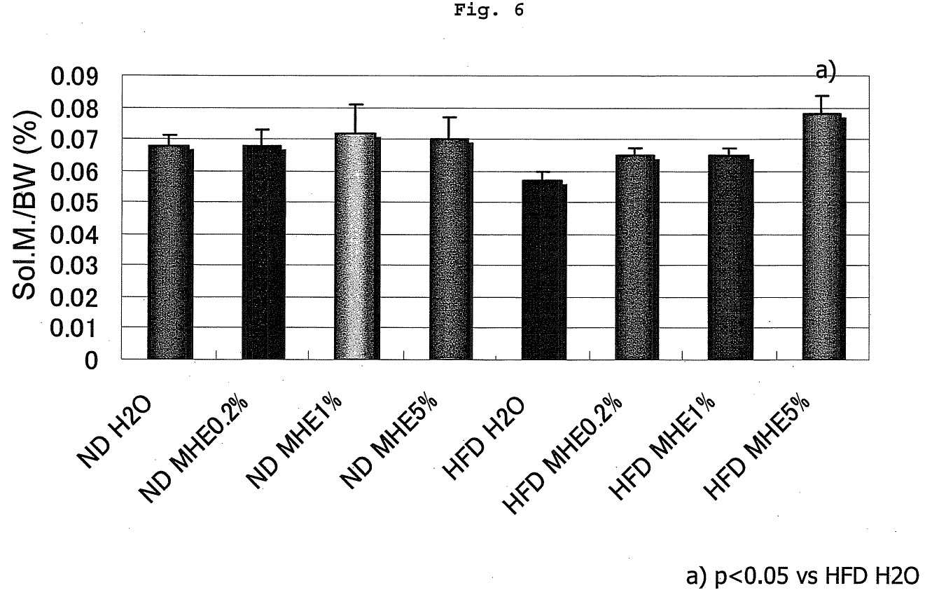

FIG. 2 shows that the body weight decreased as compared with the C1 group and the C2 group by administering the MHE. Particularly, a significant body weight decrease was observed in the B3 group. FIGS. 3 and 4 shows that the WATr and the WATt were decreased as compared with those of the C1 and C2 groups by administering the MHE. FIG. 5 shows that the BAT was increased by administering the MHE. Alternatively, FIG. 6 shows that the weight of the soleus muscle was not influenced or tended to be increased by administering the MHE.

Adipocytes have lipid droplets in cell cytoplasm, and are classified into unicellular adipocytes (white adipocytes: WATr, WATt) and multilocular adipocytes (brown adipocytes: BAT). The white adipocytes, which have large-sized lipid droplets, are stored-type cells in which nuclei and cell organelles are pressed to a side edge. It is preferable that the white adipocytes decrease in order to eliminate obesity. On the other hand, the brown adipocytes, which have a number of small-sized or middle-sized lipid droplets, are metabotropic cells having developing cell organelles, and metabolize the reserved fat into energy. When the brown adipocytes increase, the metabolic system tends to eliminate obesity.

Our results showed that the MHE had an advantage toward obesity since the MHE decreased the white adipocytes and increased the brown adipocytes. Alternatively, since the MHE has no influence on the weight of the soleus muscle or tends to increase, an adverse influence of decreasing the muscle weight to decrease the metabolic rate was not observed. Particularly, since a significant increase of the B3 group to the C2 group was observed, the B3 group showed a very ideal anti-obesity effect of increasing the brown adipocytes and the muscle weight to increase the basal metabolic rate to decrease the white adipocytes.

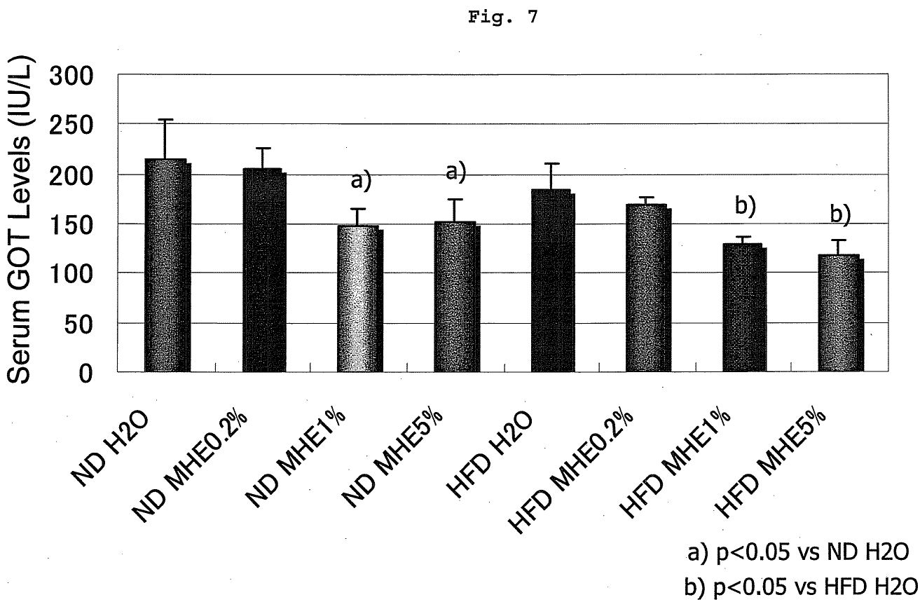

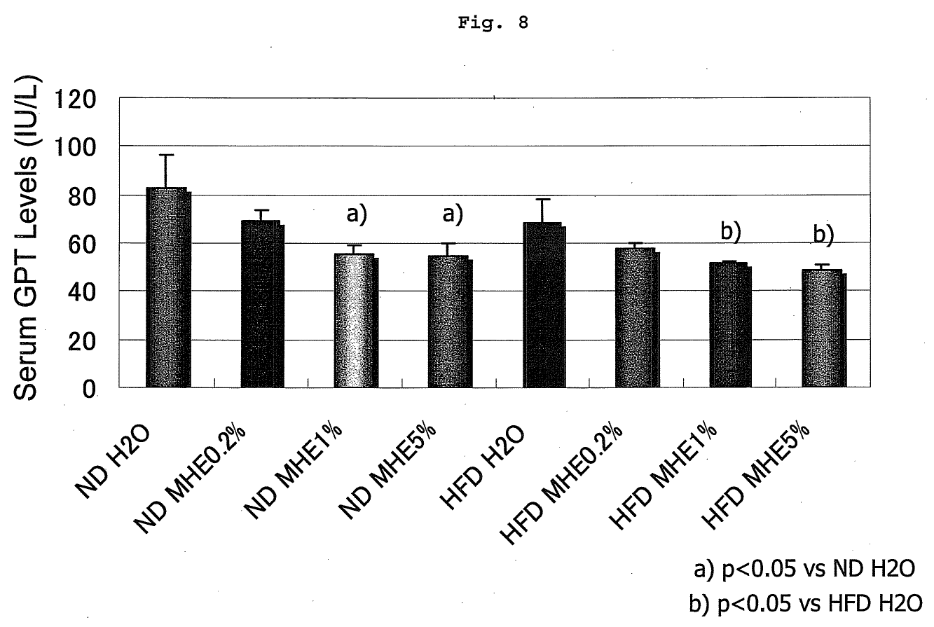

FIGS. 7 and 8 show that the serum level of GOT and GPT were decreased by administering the MHE. Particularly, the serum GOT and GPT of the A2, A3, B2 and B3 groups decreased significantly between the C1 and C2 groups. Some anti-obesity agents cause hepatic damage to increase the serum level of GOT and GPT. MHE did not exhibit such damage and had a hepatic protection function as well.

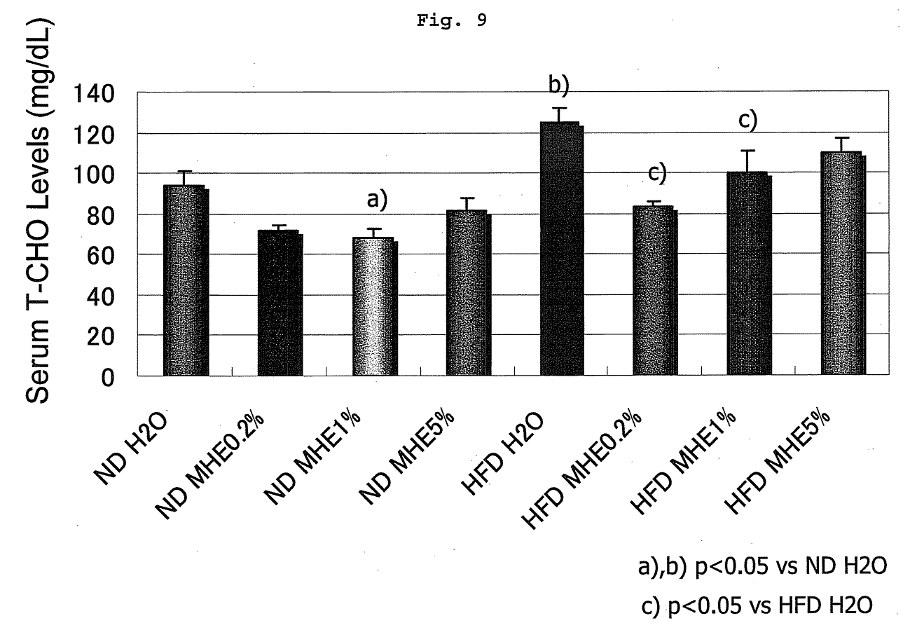

FIGS. 9 to 13 show that all parameters showing serum fat (T-CHO, TG, FFA, LDL-C, and HDL-C) showed an effective change in anti-obesity (that is, T-CHO, TG, FFA and LDL-C are decreased, and HDL-C is increased) by administering the MHE. These parameters were significantly decreased or increased between the C1 and C2 groups in some MHC administered groups.

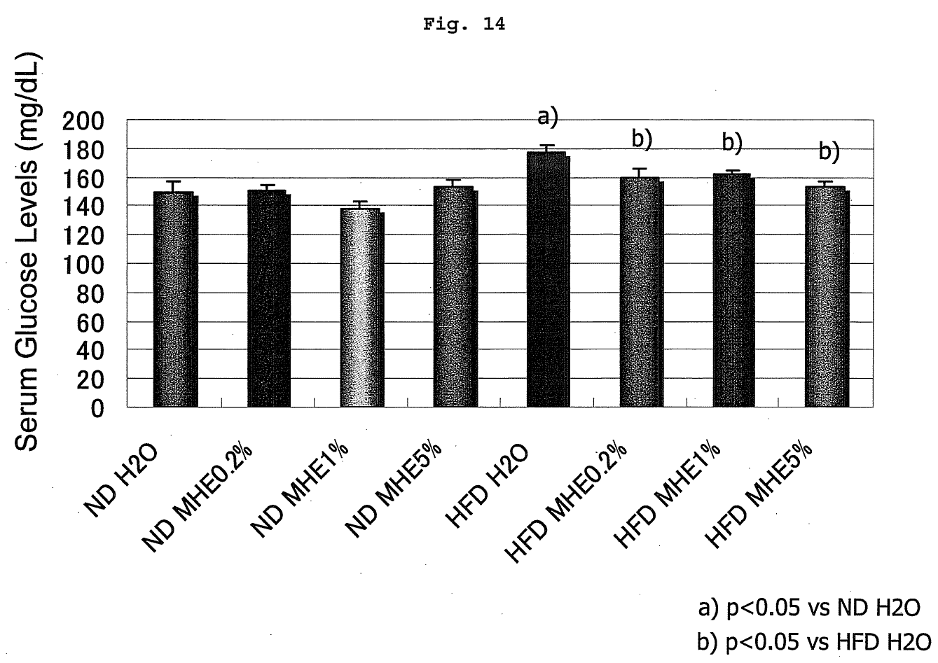

FIGS. 14 to 16 show that the MHE had no influence on the serum glucose levels or slightly decreased the levels. Alternatively, the serum insulin levels of the B1 to B3 groups decreased significantly to that of the C2 group, and the serum adiponectin levels increased significantly to the C2 group. According to these results, the MHE had no significant effect on the serum glucose levels and the serum insulin concentration in the normal diet. Furthermore rats administered MHE reacted by a slight amount of insulin, even when the serum glucose levels increased by a high fat diet (reduction in insulin resistance).

### Example 3

### Effect Confirming Test of Anti-Obesity Agent 1 (FCGr), Anti-Obesity Agent 2 (FCGf) and Anti-Obesity Agent 3 (MHE)

The effect confirming test of FCGr, FCGf, and MHE was performed using four-week old male SD rats. Four rats were used per one group from any of the following groups.

C group (positive control: HFD H20): A high fat diet (HFD) containing 35% lard was given to the rats after preliminarily breeding the rats for one week. The rats were made to freely take water (H2O) as drinking water.

D1 group (HFD FCGr 5%): The rats were made to freely take the HFD containing FCGr of 5% after preliminarily breeding the rats for one week. The rats were made to freely take water as drinking water.

D2 group (HFD FCGf 5%): The rats were made to freely take the HFD containing FCGf of 5% after preliminarily breeding the rats for one week. The rats were made to freely take water as drinking water.

D3 group (HFD MHE 5%): The rats were made to freely take the HFD containing MHE of 5% after preliminarily breeding the rats for one week. The rats were made to freely take water as drinking water.

In the above four groups, after breeding for four weeks, food intake weight per one rat per day (food intake mean (g/rat/d)), water intake volume per one rat per day (water intake mean (mL/rat/d)), body weight (g), a rate of white adipocytes surrounding the reins per body weight (WATr/BW (%)), a rate of white adipocytes surrounding the testis per body weight (WATt/BW (%)), and a rate of brown adipocytes per body weight (BAT/BW (%)) were measured.

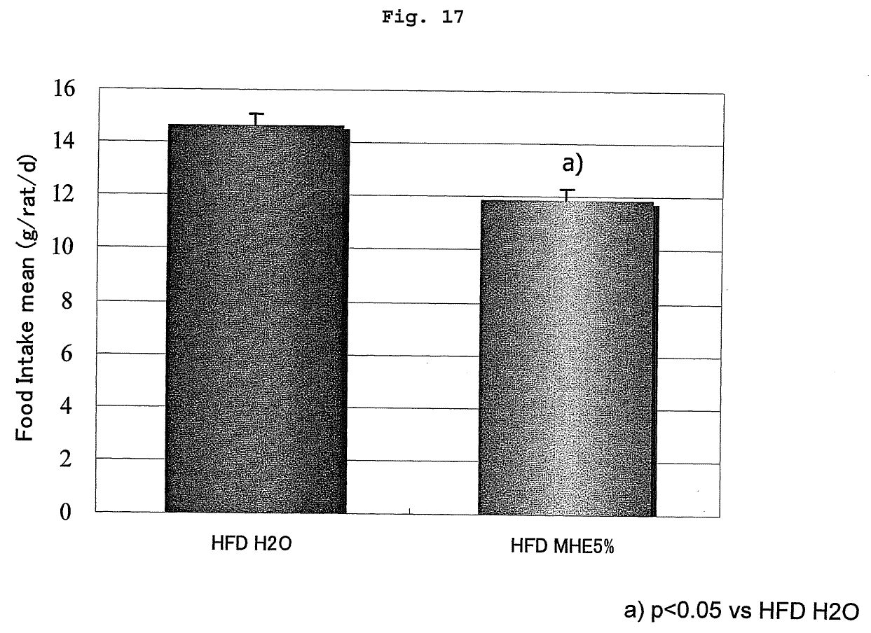

FIGS. 17 to 22 show the results of example 3. Data in the figures were shown by an average value±standard error. The symbol a) in FIGS. 17 to 22 means that a significant difference (p<0.05) was observed between the C group (HFD, H2O) and the D1 to D3 groups.

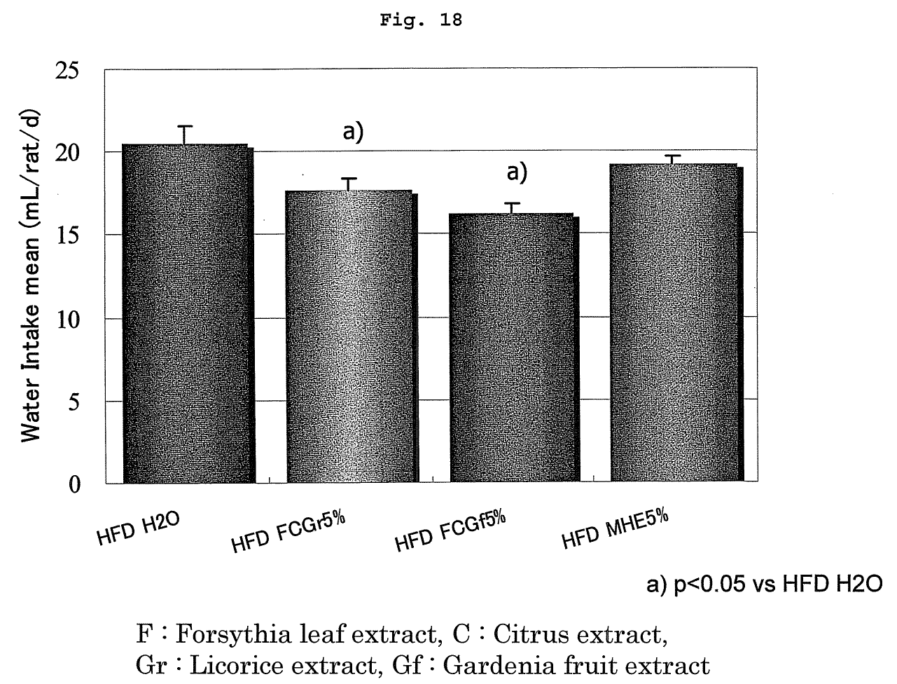

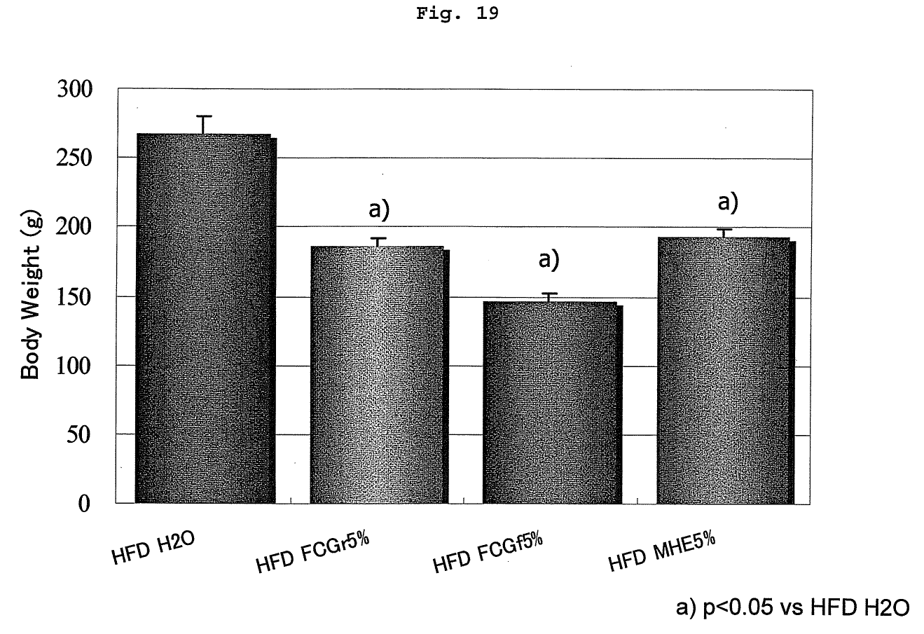

As shown in FIG. 17, the intake mean of the D3 group to which the MHE was administered significantly decreased as compared with that of the C group. As shown in FIG. 18, the water intake mean of the D1 to D3 groups to which the FCGr, the FCGf or the MHE was administered was significantly decreased as compared with that of the C group. As shown in FIG. 19, the body weight of the D1 to D3 groups significantly decreased as compared with that of the C group.

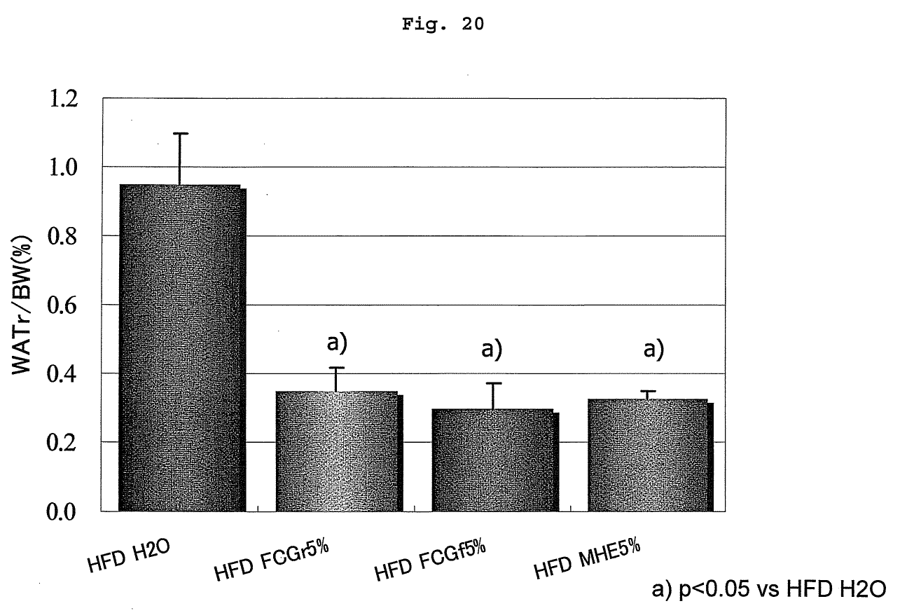

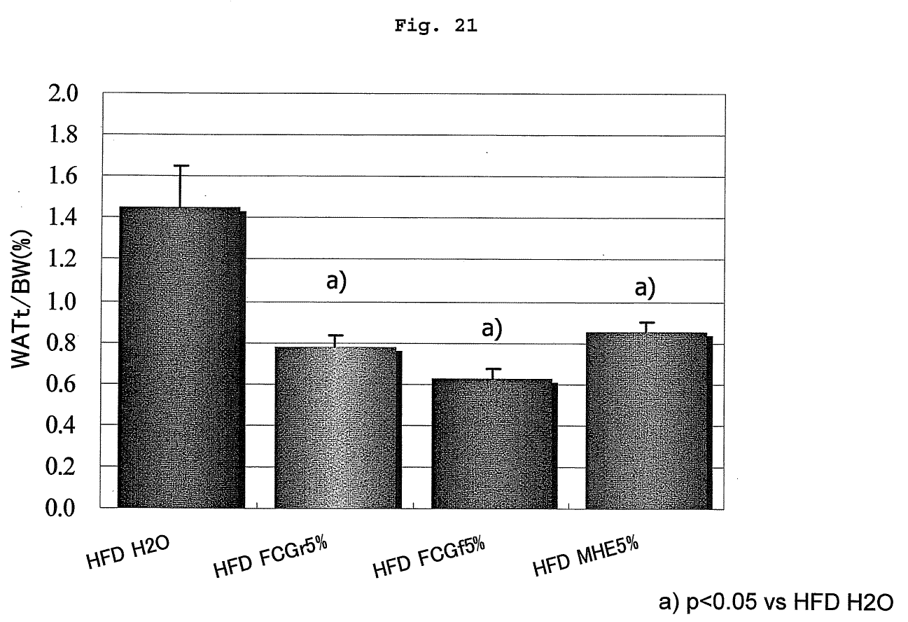

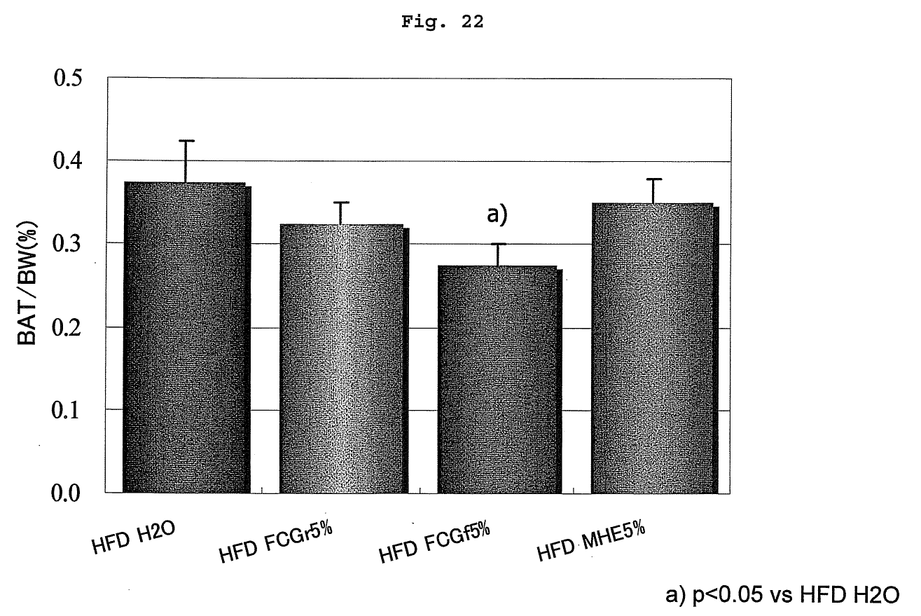

The decreases of the intake mean and water intake mean may be considered to lead to a decrease in body weight only on these data. However, FIG. 1 shows the results of the administering experiments for three months, and FIG. 17 shows the results of the administering experiments for four weeks. Therefore, an anorectic effect was observed in administering for four weeks. It was found that the long-term administration for three months showed that such an anorectic effect disappeared. As shown in FIGS. 20 and 21, the weight of the white adipocytes of the D1 to D3 groups was significantly less than that of the C group with a decrease of about 40% to 60%. On the other hand, as shown in FIG. 22, the brown adipocytes of the D1 and D3 groups do not significantly decrease as compared with those of the C group, and are considered to be almost same level.

The brown adipocytes of the D2 group significantly decreased as compared with those of the C group. However, the extent thereof was about 25%, and the rate of the decrease of the brown adipocytes was not as large as that of the white adipocytes. In example 2, it turns out that the MHE exhibits an anti-obesity effect of increasing the brown adipocytes and the muscle weight to increase the basal metabolic rate to decrease the white adipocytes. In example 3, in considering that the test period of four weeks was shorter than three months (12 weeks) of example 2 and the number of parameters was fewer than that of example 2, the anti-obesity agent of the present example was considered to exhibit an ideal anti-obesity effect of increasing the brown adipocytes and the muscle weight to increase the basal metabolic rate to decrease the white adipocytes as in the result of example 2.

### Example 4

### Formulation Example

Next, a formulation example in providing an anti-obesity agent of the present embodiment will be described.

Tablets were produced by thoroughly mixing any one of the anti-obesity agents 1 to 3 (FCGr, FCGf, or MHE) of 50 mg, lactose of 178 mg, cornstarch of 30 mg, microcrystalline cellulose of 30 mg and sucrose fatty acid ester of 3 mg and using a conventionally known tableting device (for example, LIBRA2 produced by Kikusui Seisakusho Ltd.). The weight of each of the tablets was 300 mg.

Granules were produced by thoroughly mixing any one of the anti-obesity agents 1 to 3 (FCGr, FCGf, or MHE) of 300 mg, lactose of 216 mg, microcrystalline cellulose of 60 mg and sucrose fatty acid ester of 6 mg and using a conventionally known dry granulating machine (for example, TF208 produced by Freund Corporation). The weight per one pack of the granules was 600 mg, and was set as administering granules for one-time.

Powdered medicines were produced by thoroughly mixing any one of the anti-obesity agents 1 to 3 (FCGr, FCGf, or MHE) of 300 mg, cornstarch of 180 mg and lactose of 120 mg. The weight per one pack of the powdered medicines was 600 mg, and was set as administering powdered medicines for one-time.

Liquid medicines were produced by thoroughly mixing any one of the anti-obesity agents 1 to 3 (FCGr, FCGf, or MHE) of 600 mg, glycerin of 100 mg, D-sorbitol of 100 mg, citrate of 500 mg, and sodium benzoate of 30 mg and adding refining water so that the whole amount is set to 100 mL. The amount per one bottle of the liquid medicines was 100 mL, and was set as administering liquid medicines for one-time.

Thus, the present embodiment provided the anti-obesity agent containing the extracts of three kinds (Forsythia leaf extract, Citrus extract, Licorice extract, or Forsythia leaf extract, Citrus extract, Gardenia fruit extract) or four kinds (Forsythia leaf extract, Citrus extract, Licorice extract, and the Gardenia fruit extract). Since these anti-obesity agents consist of at least one extract of Forsythia leaf, Citrus, and Licorice or Gardenia fruit, the anti-obesity agents are easily produced and provided. Alternatively, it was found that the anti-obesity agent of the present embodiment showed the ideal anti-obesity effect of increasing the brown adipocytes and the muscle weight to increase the basal metabolic rate to decrease the white adipocytes.

The present invention provides an anti-obesity agent containing three or four kinds of extracts. Since all the anti-obesity agent can also be treated as food products, the anti-obesity agent can be provided not only as a medicinal product but also as a health diet and so on.

What is claimed is:

1. An anti-obesity agent comprising Forsythia leaf extract, Citrus extract, and at least one extract selected from the group consisting of Licorice extract and Gardenia fruit extract.

2. The anti-obesity agent according to claim 1, wherein the anti-obesity agent comprises both the Licorice extract and the Gardenia fruit extract.

3. The anti-obesity agent according to claim 1, wherein the dosage form of the anti-obesity agent is any one of a tablet, a granule, a powdered medicine and a liquid medicine.

4. The anti-obesity agent according to claim 2, wherein the dosage form of the anti-obesity agent is any one of a tablet, a granule, a powdered medicine and a liquid medicine.
